(19) United States
(12) Patent Application Publication
(10) Pub. No.: US 2002/0042370 A1
(43) Pub. Date: Apr. 11, 2002

(54) Method of treating graft rejection using inhibitors of CCR2 function

(75) Inventor: Wayne W. Hancock, Medfield, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Millennium Pharmaceuticals, Inc., Cambridge, MA (US)

(21) Appl. No.: 09/835,087

(22) Filed: Apr. 13, 2001

Related U.S. Application Data
(63) Continuation-in-part of application No. 09/549,448, filed on Apr. 14, 2000, now abandoned.

(51) Int. Cl. A61K 38/17; A61K 31/00

(52) U.S. Cl. 514/12; 514/1

(57) ABSTRACT

A method for inhibiting the rejection of transplanted grafts is disclosed. The method comprising administering an effective amount of an antagonist of CCR2 function to a graft recipient. The disclosed methods can also comprise the co-administration of one or more additional therapeutic agents, for example, immunosuppressive agents.

### RELATED APPLICATION

This application is a continuation-in-part of application Ser. No. 09/549,448, filed on Apr. 14, 2000, the entire teachings of which are incorporated herein by reference.

### BACKGROUND OF THE INVENTION

In many instances, the best and only treatment available to patients suffering from certain end stage degenerative conditions or congenital genetic disorders is transplantation of a healthy graft (e.g., organs, tissues). Advances in surgical techniques and post-operative immunosuppressive therapy have mitigated some of the barriers to long-term survival of grafts and graft recipients, and ushered this once experimental therapy into wider clinical practice.

A major barrier to the long-term survival of transplanted grafts is rejection by the recipient's immune system. Graft rejection can be classified as hyper-acute rejection which is mediated by preformed antibodies that can bind to the graft and are present in the circulation of the recipient, acute rejection which is mediated by the recipient's cellular immune response or chronic rejection which occurs via a multi-factorial process that includes an immune component. The practice of matching the allelic variants of cellular antigens, most notably major histocompatibility antigens (MHC), also referred to as tissue typing, as well as matching of the blood type of the donor and recipient has reduced the incidence of hyper-acute rejection. However, most grafts which are transplanted do not exactly match the tissue type of the recipient (e.g., allografts) and will not remain viable without therapeutic intervention.

The rejection of allografts can be inhibited by long-term (e.g., life-long) prophylactic immunosuppressive therapy, most notably with agents that inhibit calcineurin (e.g., cyclosporin A (CsA), FK-506). Immunosuppressive therapy not only inhibits rejection of the graft, but can render the recipient susceptible to infection with, for example, viruses, bacteria and fungi (e.g., yeasts, molds), and at higher risk for the development of certain malignancies. Additionally, therapeutic doses of immunosuppressive agents can produce adverse side effects, such as diabetes mellitus, neurotoxicity, nephrotoxicity, hyperlipidemia, hypertension, hirsutism and gingival hyperplasia (Spencer, C. M., et al., Drugs 54(6):925-975 (1997)). Thus, the degree of immunosuppression must be carefully tailored to prevent rejection of the graft and to preserve the general health of the recipient.

Despite such prophylactic immunosuppression, the acute and chronic rejection of grafts remains a clinical problem. Acute episodes of rejection are characterized by infiltration of the graft by the recipient's leukocytes (e.g., monocytes, macrophages, T cells) and cellular necrosis. These episodes usually occur during the days to months following transplantation. Acute rejection has been treated with high doses of certain immunosuppressive agents, such as glucocorticoids (e.g., prednisone) and certain antibodies which bind to leukocytes (e.g., OKT3). However, these therapies do not always stop the rejection, are associated with systemic side effects and can lose efficacy in cases of recurrent rejection activity.

Chronic rejection becomes the major cause of graft failure and recipient death for those patients that survive past the first year. For example, evidence of chronic rejection can be found in about 40-50% of heart and/or lung allograft recipients who survive for five years, and most kidney grafts succumb to chronic rejection. The pathogenesis of chronic rejection is complex and involves accelerated arteriosclerosis (e.g., atherosclerosis) of the graft-associated vasculature and leukocyte infiltration. Unlike acute rejection episodes, chronic rejection is not generally responsive to further immunosuppressive therapy. Furthermore, the graft accelerated arteriosclerosis characteristic of chronic rejection is generally diffuse and not amenable to conventional therapeutic procedures (e.g., angioplasty, bypass grafting, endarterectomy). Thus, patients who chronically reject their grafts can require a second transplant. (Schroeder J. S. “Cardiac Transplantation”, pp. 1298-1300; Maurer, J. R. “Lung Transplantation”, pp. 1491-1493; Carpenter, C. B. and Lazarus, J. M. “Dialysis and Transplantation in the Treatment of Renal Failure”, pp. 1524-1529; Dienstag, J. “Liver Transplantation”, pp. 1721-1725; all in Harrison's Principles of Internal Medicine, 14^{th} ed., Fauci et al. Eds. McGraw Hill (1998)).

A need exists for therapeutic methods for preventing graft rejection.

### SUMMARY OF THE INVENTION

The invention relates to transplantation and to promoting the viability of transplanted grafts. In one aspect, the invention relates to a method for inhibiting (reducing or preventing) graft rejection (e.g., acute rejection, chronic rejection). In one embodiment, the method comprises administering to a graft recipient an effective amount of an antagonist of CCR2 function. In another embodiment, the graft is an allograft. In a particular embodiment, the allograft is a heart. In a preferred embodiment, the method comprises administration of an effective amount of an antagonist of CCR2 function and an effective amount of one or more immunosuppressive agents to a graft recipient.

### BRIEF DESCRIPTION OF THE DRAWINGS

FIG. 1 shows the amino acid sequences of the light chain variable region (Vκ) of murine (Mus musculus) mAb 1D9 (SEQ ID NO:1), the light chain variable region (Vκ) of human (Homo sapiens) antibody HF-21/28 (SEQ ID NO:2) and the variable regions of several humanized 1D9 light chains (1D9RK_{A} Vκ, SEQ ID NO:3; 1D9RK_{B} Vκ, SEQ ID NO:4; 1D9RK_{C} Vκ, SEQ ID NO:5; 1D⁹RK_{D} Vκ, SEQ ID NO:6; 1D9RK_{E} Vκ, SEQ ID NO:7). Where the amino acid residues of the murine 1D9 light chain variable region (SEQ ID NO:1) and the human HF-21/28 light chain variable region (SEQ ID NO:2; Kabat database ID number 005056, and Chastagner et al., Gene. 101(2):305-6 (1991), the teachings of both of which are incorporated herein by reference in their entirety) sequences match, a dot [.] is shown. Where no amino acid is present at a specific residue position a dash [-] is shown. Where an amino acid in the HF-21/28 frame work region (FR) is changed in a humanized 1D9 variable region, it is highlighted in bold. The complementarity determining regions (CDRs) (CDR1, CDR2 and CDR3) are indicated by [==L1==], [==L2==] and [==L3==]. The numbering used is according to Kabat et al., Sequences ofproteins of immunological interest, Fifth edition, U.S. Department of Health and Human Services, U.S. Government Printing Office (1991). The amino acid sequence of CDR1 of the light chain of mAb lD9 is KSSQSLLDSDGKTFLN (SEQ ID NO:14), the amino acid sequence of CDR2 is LVSKLDS (SEQ ID NO:15) and the amino acid sequence of CDR3 is WQGTHFPYT (SEQ ID NO:16).

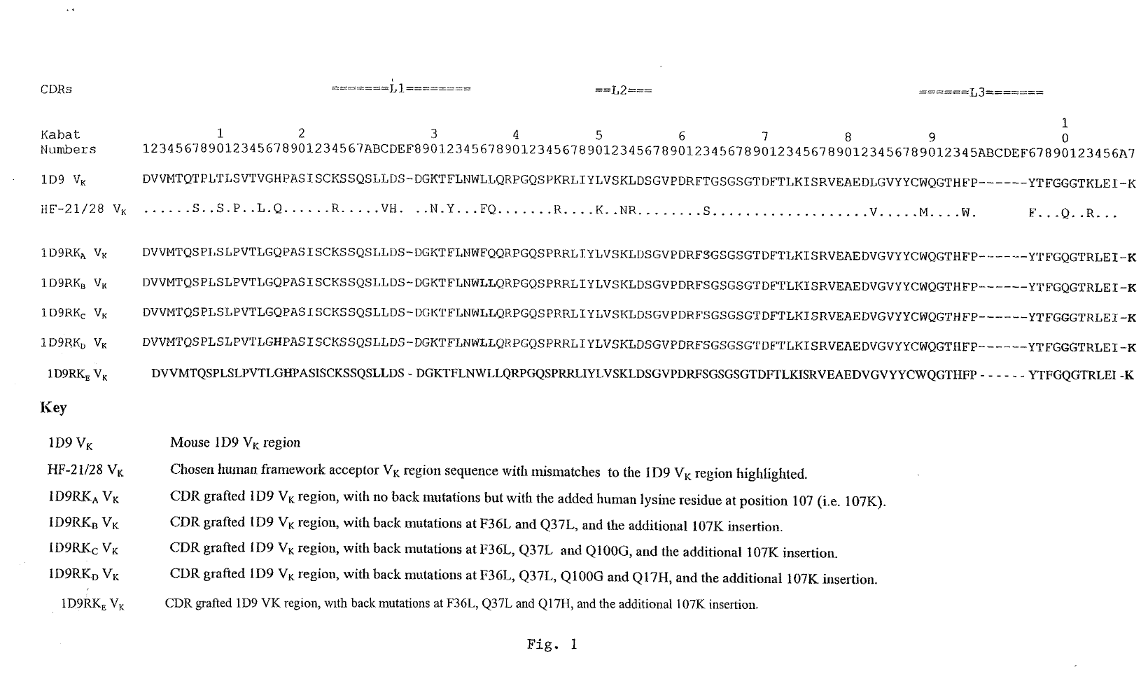

FIG. 2 shows the amino acid sequences of heavy chain variable region (V_{H}) of murine (Mus musculus) mAb 1D9 (SEQ ID NO:8), the heavy chain variable region of human (Homo sapiens) antibody 4B4′CL (SEQ ID NO:9; Kabat data base ID number 000490, and Sanz et al., Journal of Immunology. 142:883 (1989), the teachings of both of which are incorporated herein by reference in their entirety), and the variable regions of several humanized 1D9 heavy chains (1D9RH^{A} V^{H}, SEQ ID NO:10; 1D9RH_{B} V_{H}, SEQ ID NO:11; 1D9RH_{C} V_{H}, SEQ ID NO:12; 1D9RH_{D} V_{H}, SEQ ID NO:13). Where the amino acid residues of the murine 1D9 heavy chain variable region (SEQ ID NO:8) and the human 4B4′CL heavy chain variable region (SEQ ID NO:9) sequences match, a dot [.] is shown. Where no amino acid is present at a specific residue position a dash [-] is shown. Where an amino acid in the 4B4′CL heavy chain variable region is changed in a humanized 1D9 heavy chain variable region, it is highlighted in bold. The CDRs (CDR1, CDR2 and CDR3) are indicated by [−=H1==], [==H2==] and [==H3==], while [-----] denotes part of the H1 structure loop. The numbering used is according to Kabat et al., Sequences of proteins of immunological interest, Fifth edition, U.S. Department of Health and Human Services, U.S. Government Printing Office (1991). The amino acid sequence of CDR1 of the heavy chain of mAb 1D9 is AYAMN (SEQ ID NO:17), the amino acid sequence of CDR2 is RIRTKNNNYATYYADSVKD (SEQ ID NO:18) and the amino acid sequence of CDR3 is FYGNGV (SEQ ID NO:19).

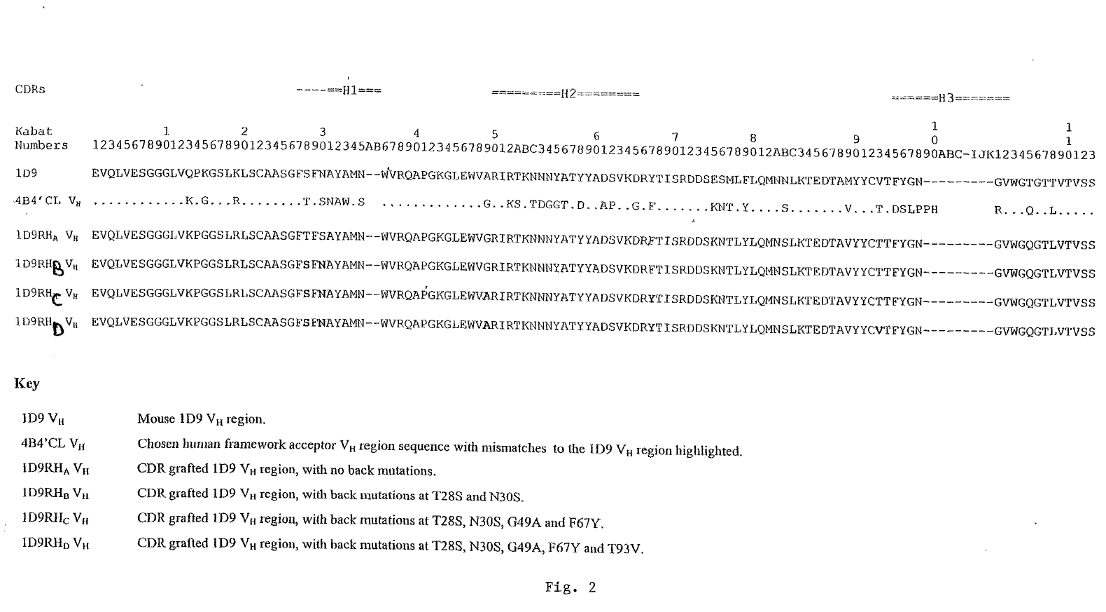

### DETAILED DESCRIPTION OF THE INVENTION

The invention relates to transplantation and to promoting the viability of transplanted grafts. Specifically, the invention relates to inhibiting graft rejection (e.g., acute graft rejection, chronic graft rejection) by administering to a graft recipient an effective amount of an antagonist of mammalian (e.g., human, Homo sapiens) CC chemokine receptor 2, CCR2.

Chemokines are a family of proinflammatory mediators that promote recruitment and activation of multiple lineages of leukocytes (e.g., lymphocytes, macrophages). They can be released by many kinds of tissue cells after activation. Continuous release of chemokines at sites of inflammation can mediate the ongoing migration and recruitment of effector cells to sites of chronic inflammation. The chemokines are related in primary structure and share four conserved cysteines, which form disulfide bonds. Based upon this conserved cysteine motif, the family can be divided into distinct branches, including the C-X-C chemokines (α-chemokines), and the C-C chemokines (β-chemokines), in which the first two conserved cysteines are separated by an intervening residue, or are adjacent residues, respectively (Baggiolini, M. and Dahinden, C. A., Immunology Today, 15:127-133 (1994)).

The C-X-C chemokines include a number of potent chemoattractants and activators of neutrophils, such as interleukin 8 (IL-8), PF4 and neutrophil-activating peptide-2 (NAP-2). The C-C chemokines include, for example, RANTES (Regulated on Activation, Normal T Expressed and Secreted), the macrophage inflammatory proteins 1 α and 1 β (MIP-1 α and MIP-1 β), eotaxin and human monocyte chemotactic proteins 1-3 (MCP-1, MCP-2, MCP-3), which have been characterized as chemoattractants and activators of monocytes or lymphocytes. Chemokines, such as IL-8, RANTES and MIP-1 α, for example, have been implicated in human acute and chronic inflammatory diseases including respiratory diseases, such as asthma and allergic disorders.

The chemokine receptors are members of a superfamily of G protein-coupled receptors (GPCR) which share structural features that reflect a common mechanism of action of signal transduction (Gerard, C. and Gerard, N. P., Annu Rev. Immunol., 12:775-808 (1994); Gerard, C. and Gerard, N. P., Curr. Opin. Immunol., 6:140-145 (1994)). Conserved features include seven hydrophobic domains spanning the plasma membrane, which are connected by hydrophilic extracellular and intracellular loops. The majority of the primary sequence homology occurs in the hydrophobic transmembrane regions with the hydrophilic regions being more diverse. The receptors for the C-C chemokines include: CCR1 which can bind, for example, MIP-1 α, RANTES, MCP-2, MCP-3, MCP-4, CKbeta8, CKbeta8-1, leukotactin-1, HCC-1 and MPIF-1; CCR2 which can bind, for example, MCP-1, MCP-2, MCP-3, MCP-4 and MCP-5; CCR3 which can bind, for example, eotaxin, eotaxin-2, RANTES, MCP-2, MCP-3 and MCP-4; CCR4 which can bind, for example, TARC, RANTES, MIP-1 α and MCP-1; CCR5 which can bind, for example, MIP-1 α, RANTES, and MIP-1; CCR6 which can bind, for example, LARC/MIP-3 α/exodus; CCR7 which can bind, for example, ELC/MIP-3 β; CCR8 which can bind, for example, 1-309; CCR9 which can bind, for example, TECK and CCR10 which can bind, for example, ESkine and CCL27 (Baggiolini, M.,Nature 392:565-568 (1998); Luster, A. D., New England Journal of Medicine, 338(7):436-445 (1998); Tsou, et al., J. Exp. Med., 188:603-608 (1998); Nardelli, et al., J Immunol, 162(1):435-444 (1999); Youn, et al., Blood, 91(9):3118-3126 (1998); Youn, et al., J Immunol, 159(11):5201-5201 (1997); Zaballos, etal., J Immunol, 162:5671-5675 (1999); Jarmin, et al., J Immunol, 164:3460-3464 (2000); Homey et al., J Immunol, 164:3465-3470 (2000)). The receptors for the CXC chemokines include: CXCR1 which can bind, for example, IL-8, GCP-2; CXCR2 which can bind, for example, IL-8, GROα/β/γ, NAP-2, ENA78, GCP-2; CXCR3 which can bind, for example, interferon gamma (IFNγ)-inducible protein of 10 kDa (IP-10), monokine induced by IFNγ (Mig), interferon-inducible T cell chemoattractant (I-TAC); CXCR4 which can bind, for example, SDF-1; and CXCR5 which can bind, for example, BCA-1I/BLC (Baggiolini M., Nature, 392:565-568 (1998); Lu et al., Eur J Immunol, 29:3804-3812 (1999)).

CCR2 as well as processes and cellular responses mediated by CCR2, are involved in rejection of transplanted grafts. As described herein, studies of allograft survival using a murine cardiac transplantation model were undertaken. Mice which lacked functional chemokine receptor CCR2 as a result of targeted disruption of the CCR2 gene (CCR2 KO mice) did not reject transplanted allografts, which were mismatched at MHC class I and MHC class II, as rapidly as control mice which had a functional CCR2 gene (CCR2+/+mice) and were otherwise genetically identical to CCR2 KO mice (see Example 1). Accordingly, a first aspect of the invention provides a method for inhibiting rejection (e.g., acute and/or chronic rejection) of a graft, comprising administering to a graft recipient an effective amount of an antagonist of CCR2 function.

CCR2 Antagonists

As used herein, the term “antagonist of CCR2 function” refers to an agent (e.g., a molecule, a compound) which can inhibit a (i.e., one or more) function of CCR2. For example, an antagonist of CCR2 function can inhibit the binding of one or more ligands (e.g., MCP-1, MCP-2, MCP-3, MCP-4) to CCR2 and/or inhibit signal transduction mediated through CCR2 (e.g., GDP/GTP exchange by CCR2 associated G proteins, intracellular calcium flux). Accordingly, CCR2-mediated processes and cellular responses (e.g., proliferation, migration, chemotactic responses, secretion or degranulation) can be inhibited with an antagonist of CCR2 function. As used herein, “CCR2” refers to naturally occurring CC chemokine receptor 2 (e.g., mammalian CCR2 (e.g., human (Homo sapiens) CCR2) and encompasses naturally occurring variants, such as allelic variants and splice variants (e.g., CC-chemokine receptor 2a and/or CC-chemokine receptor 2b).

Preferably, the antagonist of CCR2 function is a compound which is, for example, a small organic molecule, natural product, protein (e.g., antibody, chemokine, cytokine), peptide or peptidomimetic. Several molecules that can antagonize one or more functions of chemokine receptors (e.g., CCR2) are known in the art, including the small organic molecules disclosed in, for example, international patent application WO 97/24325 by Takeda Chemical Industries, Ltd.; WO 98/38167 by Pfizer, Inc.; WO 97/44329 by Teijin Limited; WO 98/04554 by Banyu Pharmaceutical Co., Ltd.; WO 98/27815, WO 98/25604, WO 98/25605, WO 98/25617 and WO 98/31364 by Merck & Co., Inc.; Hesselgesser et al., J. Biol. Chem. 273(25):15687-15692 (1998); and Howard et al., J. Medicinal Chem. 41(13):2184-2193 (1998); proteins, such as antibodies (e.g., polyclonal sera, monoclonal, chimeric, humanized, human) and antigen-binding fragments thereof (e.g., Fab, Fab′, F(ab′)₂, Fv), for example, those disclosed in WO 00/05265 by LeukoSite, Inc.; chemokine mutants and analogues, for example, those disclosed in U.S. Pat. No. 5,739,103 issued to Rollins et al., WO 96/38559 by Dana Farber Cancer Institute and WO 98/06751 by Research Corporation Technologies, Inc.; peptides, for example, those disclosed in WO 98/09642 by The United States of America. The entire teachings of each of the above cited patent applications and references are incorporated herein by reference.

Antagonists of CCR2 flnction can be identified, for example, by screening libraries or collections of molecules, such as, the Chemical Repository of the National Cancer Institute, as described herein or using other suitable methods.

Another source of antagonists of CCR2 function are combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods, such as the methods described herein.

The term “natural product”, as used herein, refers to a compound which can be found in nature, for example, naturally occurring metabolites of marine organisms (e.g., tunicates, algae), plants or other organisms and which possess biological activity, e.g., can antagonize CCR2 function. For example, lactacystin, paclitaxel and cyclosporin A are natural products which can be used as anti-proliferative or immunosuppressive agents.

Natural products can be isolated and identified by suitable means. For example, a suitable biological source (e.g., vegetation) can be homogenized (e.g., by grinding) in a suitable buffer and clarified by centrifugation, thereby producing an extract. The resulting extract can be assayed for the capacity to antagonize CCR2 function, for example, by the assays described herein. Extracts which contain an activity that antagonizes CCR2 function can be further processed to isolate the CCR2 antagonist by suitable methods, such as, fractionation (e.g., column chromatography (e.g., ion exchange, reverse phase, affinity), phase partitioning, fractional crystallization) and assaying for biological activity (e.g., antagonism of CCR2 activity). Once isolated the structure of a natural product can be determined (e.g., by nuclear magnetic resonance (NMR)) and those of skill in the art can devise a synthetic scheme for synthesizing the natural product. Thus, a natural product can be isolated (e.g., substantially purified) from nature or can be fully or partially synthetic. A natural product can be modified (e.g., derivatized) to optimize its therapeutic potential. Thus, the term “natural product”, as used herein, includes those compounds which are produced using standard medicinal chemistry techniques to optimize the therapeutic potential of a compound which can be isolated from nature.

The term “peptide”, as used herein, refers to a compound consisting of from about two to about ninety amino acid residues wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook, J. et al., Molecular Cloning: A Laboratory Manual, Cold Spring Harbor Press, Cold Spring Harbor, N.Y. (1989)). A “peptide” can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are know in the art and are disclosed in, for example, Green and Wuts, “Protecting Groups in Organic Synthesis”, John Wiley and Sons, 1991. The functional groups of a peptide can also be derivatized (e.g., alkylated) using art-known methods.

Peptides can be synthesized and assembled into libraries comprising a few to many discrete molecular species. Such libraries can be prepared using well-known methods of combinatorial chemistry, and can be screened as described herein or using other suitable methods to determine if the library comprises peptides which can antagonize CCR2 function. Such peptide antagonists can then be isolated by suitable methods.

The term “peptidomimetic”, as used herein, refers to molecules which are not polypeptides, but which mimic aspects of their structures. For example, polysaccharides can be prepared that have the same functional groups as peptides which can antagonize CCR2. Peptidomimetics can be designed, for example, by establishing the three dimensional structure of a peptide agent in the environment in which it is bound or will bind to CCR2. The peptidomimetic comprises at least two components, the binding moiety or moieties and the backbone or supporting structure.

The binding moieties are the chemical atoms or groups which will react or form a complex (e.g., through hydrophobic or ionic interactions) with CCR2, for example, with the amino acid(s) at or near the ligand binding site. For example, the binding moieties in a peptidomimetic can be the same as those in a peptide antagonist of CCR2. The binding moieties can be an atom or chemical group which reacts with the receptor in the same or similar manner as the binding moiety in a peptide antagonist of CCR2. Examples of binding moieties suitable for use in designing a peptidomimetic for a basic amino acid in a peptide are nitrogen containing groups, such as amines, ammoniums, guanidines and amides or phosphoniums. Examples of binding moieties suitable for use in designing a peptidomimetic for an acidic amino acid can be, for example, carboxyl, lower alkyl carboxylic acid ester, sulfonic acid, a lower alkyl sulfonic acid ester or a phosphorous acid or ester thereof.

The supporting structure is the chemical entity that, when bound to the binding moiety or moieties, provides the three dimensional configuration of the peptidomimetic. The supporting structure can be organic or inorganic. Examples of organic supporting structures include polysaccharides, polymers or oligomers of organic synthetic polymers (such as, polyvinyl alcohol or polylactide). It is preferred that the supporting structure possess substantially the same size and dimensions as the peptide backbone or supporting structure. This can be determined by calculating or measuring the size of the atoms and bonds of the peptide and peptidomimetic. In one embodiment, the nitrogen of the peptide bond can be substituted with oxygen or sulfur, thereby forming a polyester backbone. In another embodiment, the carbonyl can be substituted with a sulfonyl group or sulfinyl group, thereby forming a polyamide (e.g., a polysulfonamide). Reverse amides of the peptide can be made (e.g., substituting one or more —CONH—groups for a —NHCO—group). In yet another embodiment, the peptide backbone can be substituted with a polysilane backbone.

These compounds can be manufactured by known methods. For example, a polyester peptidomimetic can be prepared by substituting a hydroxyl group for the corresponding α-amino group on amino acids, thereby preparing a hydroxyacid and sequentially esterifying the hydroxyacids, optionally blocking the basic and acidic side chains to minimize side reactions. An appropriate chemical synthesis route can generally be readily identified upon determining the desired chemical structure of the peptidomimetic.

Peptidomimetics can be synthesized and assembled into libraries comprising a few to many discrete molecular species. Such libraries can be prepared using well-known methods of combinatorial chemistry, and can be screened as described herein to determine if the library comprises one or more peptidomimetics which antagonize CCR2 function. Such peptidomimetic antagonists can then be isolated by suitable methods.

In one embodiment, the CCR2 antagonist is an antibody or antigen-binding fragment thereof having specificity for CCR2. The antibody can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. The term “antibody” as used herein also encompasses functional fragments of antibodies, including fragments of chimeric, humanized, human, primatized, veneered or single chain antibodies. Functional fragments include antigen-binding fragments which bind to CCR2. For example, antibody fragments capable of binding to CCR2 or portions thereof, including, but not limited to Fv, Fab, Fab′ and F(ab′)₂ fragments can be used. Such fragments can be produced by nzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH₁ domain and hinge region of the heavy chain. Single chain antibodies, and chimeric, human, humanized or primatized (CDR-grafted), or veneered antibodies, as well as chimeric, CDR-grafted or veneered single chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 BI; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 Bi; and Padlan, E. A. etal., EP 0 519 596 al. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M., et al., Nucl. Acids Res., 17: 5404 (1989); Sato, K., et al., Cancer Research, 53: 851-856 (1993); Daugherty, B. L. et al., Nucleic Acids Res., 19(9):2471-2476 (1991); and Lewis, A. P. and J. S. Crowe, Gene, 101: 297-302 (1991)). Using these or other suitable methods, variants can also be readily produced. In one embodiment, cloned variable regions can be mutated, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993).

Antibodies which are specific for mammalian (e.g., human) CCR2 can be raised against an appropriate immunogen, such as isolated and/or recombinant human CCR2 or portions thereof (including synthetic molecules, such as synthetic peptides). Antibodies can also be raised by immunizing a suitable host (e.g., mouse) with cells that express CCR2, such as activated T cells (see e.g., U.S. Pat. No. 5,440,020, the entire teachings of which are incorporated herein by reference). In addition, cells expressing recombinant CCR2 such as transfected cells, can be used as immunogens or in a screen for antibody which binds receptor (See e.g., Chuntharapai et al, J. Immunol., 152:1783-1789 (1994); Chuntharapai et al., U.S. Pat. No. 5,440,021).

Preparation of immunizing antigen, and polyclonal and monoclonal antibody production can be performed using any suitable technique. A variety of methods have been described (see e.g., Kohler et al., Nature, 256: 495-497 (1975) and Eur. J Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer ‘94), Ausubel, F. M. et al., Eds., (John Wiley & Sons: New York, N.Y.), Chapter 11, (1991)). When a monoclonal antibody is desired a hybridoma can generally be produced by fusing a suitable immortal cell line (e.g., a myeloma cell line such as SP2/0 or P3X63Ag8.653) with antibody producing cells. The antibody producing cells, preferably those obtained from the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies of the requisite specificity can be used, including, for example, methods which select recombinant antibody from a library (e.g., a phage display library). Transgenic animals capable of producing a repertoire of human antibodies (e.g., XenoMouse™ (Abgenix, Fremont, CA)) can be produced using suitable methods (see e.g., WO 98/24893 (Abgenix), published Jun. 11, 1998; Kucherlapate, R. and Jakobovits, A., U.S. Pat. No. 5,939,598; Jakobovits et al., Proc. Natl. Acad. Sci. USA, 90: 2551-2555 (1993); Jakobovits et al., Nature, 362: 255-258 (1993)). Additional methods for production of transgenic animals capable of producing a repertoire of human antibodies have been described (e.g., Lonberg et al., U.S. Pat. No. 5,545,806; Surani et al., U.S. Pat. No. 5,545,807; Lonberg et al., WO97/13852).

In one embodiment, the antibody or antigen-binding fragment thereof has specificity for a mammalian CC chemokine receptor 2 (CCR2), such as human CCR2. In a preferred embodiment, the antibody or antigen-binding fragment can inhibit binding of a ligand (i.e., one or more ligands) to CCR2 and/or one or more functions mediated by CCR2 in response to ligand binding. Preferred antibody antagonists of CCR2 function are disclosed in WO 00/05265 (LeukoSite, Inc.) published Feb. 3, 2000, and co-pending U.S. patent application Ser. No. 09/497,625, filed Feb. 3, 2000, the teachings of both of which are incorporated herein by reference in their entirety.

Other preferred antibodies bind mammalian CCR2 (e.g., human CCR2) and inhibit the binding of a ligand (e.g., MCP-1, MCP-2, MCP-3, MCP-4, MCP-5) to the receptor. Murine monoclonal antibodies designated 1D9 (also referred to as LS132.1D9 or 1D9-2-121-3-6) and 8G2 (also referred to as LS132.8G2), which bind CCR2 and inhibit the binding of ligand to the receptor, were produced as described herein. Hybridoma cell lines producing the antibodies were deposited on Jul. 17, 1998, on behalf of LeukoSite, Inc., 215 First Street, Cambridge, Mass. 02142, U.S.A., (now Millennium Pharmaceuticals, Inc., 75 Sidney Street, Cambridge, Mass. 02139, U.S.A.) at the American Type Culture Collection, 10801 University Boulevard, Manassas, Va. 20110, U.S.A., under Accession Nos. HB-12549 (1D9) and HB-12550 (8G2). These antibodies and, for example, chimeric or humanized versions of the antibodies can be administered in accordance with the method of the invention.

An antibody which binds CCR2 and inhibits the binding of a ligand (e.g., MCP-1, MCP-2, MCP-3, MCP-4, MCP-5) to the receptor can comprise a humanized lD9 light chain comprising an amino acid sequence selected from the group consisting of SEQ ID NO:3, SEQ ID NO:4, SEQ ID NO:5, SEQ ID NO:6 and SEQ ID NO:7, and/or a humanized 1D9 heavy chain comprising an amino acid sequence selected from the group consisting of SEQ ID NO:10, SEQ ID NO:11, SEQ ID NO:12 and SEQ ID NO:13. In certain embodiments, an antibody which binds CCR2 and inhibits the binding of a ligand to the receptor can comprise a humanized chain (e.g., a humanized 1D9 light chain comprising an amino acid sequence selected from the group consisting of SEQ ID NO:3, SEQ ID NO:4, SEQ ID NO:5, SEQ ID NO:6 and SEQ ID NO:7, or a humanized 1D9 heavy chain comprising an amino acid sequence selected from the group consisting of SEQ ID NO:10, SEQ ID NO:11, SEQ ID NO:12 and SEQ ID NO:13) and a complementary chain (heavy or light as appropriate) which is, for example, human, nonhuman (e.g., rodent (e.g., murine), primate), humanized or chimeric. A complementary light or heavy chain is one which is capable of associating with a selected heavy or light chain, respectively, resulting in an antibody or antigen- binding fragment which binds CCR2 and inhibits the binding of a ligand (e.g., MCP-1, MCP-2, MCP-3, MCP-4, MCP-5) to the receptor. Antigen-binding fragments of such antibodies (e.g., Fab fragments, F(ab′)₂ fragments, Fab′ fragments, Fv fragments) can also be administered in accordance with the method of the invention.

In certain embodiments, a humanized antibody which binds CCR2 and inhibits the binding of a ligand (e.g., MCP-1, MCP-2, MCP-3, MCP-4, MCP-5) to the receptor is administered. In particular embodiments, the humanized antibody can comprise a light chain comprising the amino acid sequence of SEQ ID NO:3 and a heavy chain comprising an amino acid sequence selected from the group consisting of SEQ ID NO:10, SEQ ID NO:11, SEQ ID NO:12 and SEQ ID NO:13. In other embodiments, the humanized antibody can comprise a light chain comprising the amino acid sequence of SEQ ID NO:4 and a heavy chain comprising an amino acid sequence selected from the group consisting of SEQ ID NO:10, SEQ ID NO:11, SEQ ID NO:12 and SEQ ID NO:13. In other embodiments, the humanized antibody which binds CCR2 and inhibits the binding of a ligand to the receptor can comprise a light chain comprising the amino acid sequence of SEQ ID NO:5 and a heavy chain comprising an amino acid sequence selected from the group consisting of SEQ ID NO:10, SEQ ID NO:11, SEQ ID NO:12 and SEQ ID NO:13. In other embodiments, the humanized antibody can comprise a light chain comprising the amino acid sequence of SEQ ID NO:6 and a heavy chain comprising an amino acid sequence selected from the group consisting of SEQ ID NO:10, SEQ ID NO:11, SEQ ID NO:12 and SEQ ID NO:13. In further embodiments, the humanized antibody can comprise a light chain comprising the amino acid sequence of SEQ ID NO:7 and a heavy chain comprising an amino acid sequence selected from the group consisting of SEQ ID NO:10, SEQ ID NO:11, SEQ ID NO:12 and SEQ ID NO:13.

In additional embodiments, the humanized antibody which binds CCR2 and inhibits the binding of a ligand (e.g., MCP-1, MCP-2, MCP-3, MCP-4, MCP-5) to the receptor can comprise a heavy chain comprising the amino acid sequence of SEQ ID NO:10 and a light chain comprising an amino acid sequence selected from the group consisting of SEQ ID NO:3, SEQ ID NO:4, SEQ ID NO:5, SEQ ID NO:6 and SEQ ID NO:7. In other embodiments, the humanized antibody can comprise a heavy chain comprising the amino acid sequence of SEQ ID NO:11 and a light chain comprising an amino acid sequence selected from the group consisting of SEQ ID NO:3, SEQ ID NO:4, SEQ ID NO:5, SEQ ID NO:6 and SEQ ID NO:7. In other embodiments, the humanized antibody can comprise a heavy chain comprising the amino acid sequence of SEQ ID NO:12 and a light chain comprising an amino acid sequence selected from the group consisting of SEQ ID NO:3, SEQ ID NO:4, SEQ ID NO:5, SEQ ID NO:6 and SEQ ID NO:7. In further embodiments, the humanized antibody which binds CCR2 and inhibits the binding of a ligand (e.g., MCP-1, MCP-2, MCP-3, MCP-4, MCP-5) to the receptor can comprise a heavy chain comprising the amino acid sequence of SEQ ID NO:13 and a light chain comprising an amino acid sequence selected from the group consisting of SEQ ID NO:3, SEQ ID NO:4, SEQ ID NO:5, SEQ ID NO:6 and SEQ ID NO:7.

In additional embodiments, the antibody which binds CCR2 and inhibits the binding of a ligand (e.g., MCP-1, MCP-2, MCP-3, MCP-4, MCP-5) to the receptor can comprise a light chain comprising the variable region of murine antibody 1D9 (SEQ ID NO:1) and a complementary heavy chain, for example, a heavy chain comprising a variable region having an amino acid sequence selected from the group consisting of SEQ ID NO:10,SEQ ID NO:11,SEQ ID NO:12 and SEQ ID NO:13. In further embodiments, the antibody which binds CCR2 and inhibits the binding of a ligand (e.g., MCP-1, MCP-2, MCP-3, MCP-4, MCP-5) to the receptor can comprise a heavy chain comprising the variable region of murine antibody 1D9 (SEQ ID NO:8) and a complementary light chain, for example, a light chain comprising a variable region having an amino acid sequence selected from the group consisting of SEQ ID NO:3, SEQ ID NO:4, SEQ ID NO:5, SEQ ID NO:6 and SEQ ID NO:7.

A preferred antibody or antigen-binding fragment thereof that can be administered to inhibit graft rejection (e.g., acute rejection, chronic rejection) in accordance with the invention can be a humanized 1D9 antibody or antigen binding fragment thereof, comprising a light chain comprising the amino acid sequence of SEQ ID NO:3 and a heavy chain comprising the amino acid sequence of SEQ ID NO:10.

Antibodies, including human, humanized and chimeric antibodies and the like, which bind CCR2 ligand (e.g., MCP-1, MCP-2, MCP-3, MCP-4, MCP-5) and inhibit binding of ligand to CCR2 can be prepared using suitable method, such as the methods described herein.

Assessment of Activity of Antagonists

The capacity of an agent (e.g., proteins, peptides, natural products, small organic molecules, peptidomimetics) to antagonize CCR2 function can be determined using a suitable screen (e.g., high through-put assay). For example, an agent can be tested in an extracellular acidification assay, calcium flux assay, ligand binding assay or chemotaxis assay (see, for example, Hesselgesser et al., J Biol. Chem. 273(25):15687-15692 (1998); WO 00/05265 and WO 98/02151).

In a particular assay, membranes can be prepared from cells which express CCR2, such as THP-1 cells (American Type Culture Collection, Manassas, VA; Accession No. TIB202) or cells which express recombinant CCR2. Cells can be harvested by centrifugation, washed twice with PBS (phosphate-buffered saline), and the resulting cell pellets frozen at −70 to −85° C. The frozen pellet can be thawed in ice-cold lysis buffer consisting of 5 mM HEPES (N-2-hydroxyethylpiperazine-N′-2-ethane-sulfonic acid) pH 7.5, 2 mM EDTA (ethylenediaminetetraacetic acid), 5 μg/ml each aprotinin, leupeptin, and chymostatin (protease inhibitors), and 100 μg/ml PMSF (phenyl methane sulfonyl fluoride—also a protease inhibitor), at a concentration of 1 to 5×10⁷ cells/ml, to achieve cell lysis. The resulting suspension can be mixed well to resuspend all of the frozen cell pellet. Nuclei and cell debris can be removed by centrifugation of 400 x g for 10 minutes at 4° C. The resulting supernatant can be transferred to a fresh tube and the membrane fragments can be collected by centrifugation at 25,000 x g for 30 minutes at 4° C. The resulting supernatant can be aspirated and the pellet can be resuspended in freezing buffer consisting of 10 mM HEPES pH 7.5, 300 mM sucrose, 1 μg/ml each aprotinin, leupeptin, and chymostatin, and 10 μg/ml PMSF (approximately 0.1 ml per each 10⁸ cells). All clumps can be resolved using a minihomogenizer, and the total protein concentration can be determined by suitable methods (e.g., Bradford assay, Lowery assay). The membrane solution can be divided into aliquots and frozen at −70 to −85° C. until needed.

The membrane preparation described above can be used in a suitable binding assay. For example, membrane protein (2 to 20 μg total membrane protein) can be incubated with 0.1 to 0.2 nM ¹²⁵I-labeled MCP-1, ¹²⁵I-labeled MCP-2, ¹²⁵I-labeled MCP-3 or ¹²⁵I-labeled MCP-4 with or without unlabeled competitor (MCP-1, MCP-2,MCP-3 and/or MCP-4) or various concentrations of compounds to be tested. ¹²⁵I-labeled MCP-1, ¹²⁵I-labeled MCP-2, ¹²⁵I-labeled MCP-3 or ¹²⁵I-labeled MCP-4 can be prepared by suitable methods or purchased from commercial vendors (e.g., DuPont-NEN (Boston, Mass.)). The binding reactions can be performed in 60 to 100 μl of a binding buffer consisting of 10 mM HEPES pH 7.2, 1 mM CaCi₂, 5 mM MgCl₂, and 0.5% BSA (bovine serum albumin), for 60 min at room temperature. The binding reactions can be terminated by harvesting the membranes by rapid filtration through glass fiber filters (e.g., GF/B or GF/C, Packard) which can be presoaked in 0.3% polyethyleneimine. The filters can be rinsed with approximately 600 μl of binding buffer containing 0.5 M NaCl, dried, and the amount of bound radioactivity can be determined by scintillation counting.

The CCR2 antagonist activity of test agents (e.g., compounds) can be reported as the inhibitor concentration required for 50% inhibition (IC₅₀ values) of specific binding in receptor binding assays (e.g., using ¹²⁵1I-labeled MCP-1, ¹²⁵I-labeled MCP-2, 125I-labeled MCP-3 or ¹²⁵I-labeled MCP-4 as ligand and membranes prepared from THP-1 cells). Specific binding is preferably defined as the total binding (e.g., total cpm on filters) minus the non-specific binding. Non-specific binding is defined as the amount of cpm still detected in the presence of excess unlabeled competitor (e.g., MCP-1, MCP-2, MCP-3, MCP-4). If desired, membranes prepared from cells which express recombinant CCR2 can be used in the described assay.

The capacity of compounds to antagonize CCR2 function can also be determined in a leukocyte chemotaxis assay using suitable cells. Suitable cells include, for example, cell lines, recombinant cells or isolated cells which express CCR2 and undergo CCR2 ligand-induced (e.g., MCP-1, MCP-2, MCP-3, MCP-4, MCP-5) chemotaxis. In one example, CCR2-expressing recombinant Li.2 cells (see Campbell, et al. J Cell Biol, 134:255-266 (1996)) or peripheral blood mononuclear cells, can be used in a modification of a transendothelial migration assay (Carr, M. W., et al., Proc. Natl Acad Sci, USA, (91):3652 (1994)). Peripheral blood mononuclear cells can be isolated from whole blood by suitable methods, for example, density gradient centrifugation and positive or preferably negative selection with specific antibodies. The endothelial cells used in this assay are preferably the endothelial cell line, ECV 304, obtained from the European Collection of Animal Cell Cultures (Porton Down, Salisbury, U.K.). Endothelial cells can be cultured on 6.5 mm diameter Transwell culture inserts (Costar Corp., Cambridge, MA) with 3.0 μm pore size. Culture media for the ECV 304 cells can consist of M199+10% FCS, L-glutamine, and antibiotics. The assay media can consist of equal parts RPMI 1640 and Ml99 with 0.5% BSA. Two hours before the assay, 2×10⁵ ECV 304 cells can be plated onto each insert of the 24 well Transwell chemotaxis plate and incubated at 37° C. Chemotactic factors such as MCP-1, MCP-2, MCP-3 or MCP-4 (commercially available from Peprotech, Rocky Hill, NJ, for example) diluted in assay medium can be added to the 24-well tissue culture plates in a final volume of 600 μL. Endothelial-coated Transwells can be inserted into each well and 106 cells of the leukocyte type being studied are added to the top chamber in a final volume of 100 μL of assay medium. The plate can then be incubated at 37° C. in 5% CO₂/95% air for 1-2 hours. The cells that migrate to the bottom chamber during incubation can be counted, for example using flow cytometry. To count cells by flow cytometry, 500 μL of the cell suspension from the lower chamber can be placed in a tube and relative counts can obtained for a set period of time, for example, seconds. This counting method is highly reproducible and allows gating on the leukocytes and the exclusion of debris or other cell types from the analysis. Alternatively, cells can be counted with a microscope. Assays to evaluate chemotaxis inhibitors can be performed in the same way as control experiment described above, except that antagonist solutions, in assay media containing up to 1% of DMSO co-solvent, can be added to both the top and bottom chambers prior to addition of the cells. Antagonist potency can be determined by comparing the number of cell that migrate to the bottom chamber in wells which contain antagonist, to the number of cells which migrate to the bottom chamber in control wells. Control wells can contain equivalent amounts of DMSO, but no antagonist. If desired, the endothelial cells can be omitted from the described chemotaxis assay and ligand-induced migration across the Transwell insert can be measured.

The activity of an antagonist of CCR2 function can also be assessed by monitoring cellular responses induced by active receptor, using suitable cells expressing receptor. For instance, exocytosis (e.g., degranulation of cells leading to release of one or more enzymes or other granule components, such as esterases (e.g., serine esterases), perforin, and/or granzymes), inflammatory mediator release (such as release of bioactive lipids such as leukotrienes (e.g., leukotriene C₄)), and respiratory burst, can be monitored by methods known in the art or other suitable methods (see e.g., Taub, D.D. et al., J. Immunol., 155: 3877-3888 (1995), regarding assays for release of granule-derived serine esterases; Loetscher et al., J. Immunol., 156: 322-327 (1996), regarding assays for enzyme and granzyme release; Rot, A. et al., J. Exp. Med., 176: 1489-1495 (1992) regarding respiratory burst; Bischoff, S. C. et al., Eur. J. Immunol., 23: 761-767 (1993) and Baggliolini, M. and C. A. Dahinden, Immunology Today, 15: 127-133 (1994)).

In one embodiment, an antagonist of CCR2 is identified by monitoring the release of an enzyme upon degranulation or exocytosis by a cell capable of this function. Cells expressing CCR2 can be maintained in a suitable medium under suitable conditions, and degranulation can be induced. The cells are contacted with an agent to be tested, and enzyme release can be assessed. The release of an enzyme into the medium can be detected or measured using a suitable assay, such as in an immunological assay, or biochemical assay for enzyme activity.

The medium can be assayed directly, by introducing components of the assay (e.g., substrate, co-factors, antibody) into the medium (e.g., before, simultaneous with or after the cells and agent are combined). The assay can also be performed on medium which has been separated from the cells or further processed (e.g., fractionated) prior to assay. For example, convenient assays are available for enzymes, such as serine esterases (see e.g., Taub, D. D. et al., J. Immunol., 155: 3877-3888 (1995) regarding release of granule-derived serine esterases).

In another embodiment, cells expressing CCR2 are combined with a ligand of CCR2 or promoter of CCR2 function, an agent to be tested is added before, after or simultaneous therewith, and degranulation is assessed. Inhibition of ligand- or promoter-induced degranulation is indicative that the agent is an inhibitor of mammalian CCR2 function.

In a preferred embodiment, the antagonist of CCR2 function does not significantly inhibit the function of other chemokine receptors (e.g., CCR1, CXCR1, CCR3). Such CCR2-specific antagonists can be identified by suitable methods, such as by suitable modification of the methods described herein. For example, cells which do not express CCR2 (CCR2⁻) but do express one or more other chemokine receptors (e.g., CCR5, CXCR1, CCR9) can be made or identified using suitable methods (e.g., transfection, antibody staining, western blot, RNAse protection). Such cells or cellular fractions (e.g., membranes) obtained from such cells can be used in a suitable binding assay. For example, when a cell which is CCR2⁻ and CCR5⁺ is chosen, the CCR2 antagonist can be assayed for the capacity to inhibit the binding of a suitable CCR5 ligand (e.g., RANTES, MIP-1 α) to the cell or cellular fraction, as described herein.

In another preferred embodiment, the antagonist of CCR2 function is an agent which binds to CCR2. Such CCR2-binding antagonists can be identified by suitable methods, for example, in binding assays employing a labeled (e.g., enzymatically labeled (e.g., alkaline phosphatase, horse radish peroxidase), biotinylated, radio-labeled (e.g., ³H, ¹⁴C, ¹²⁵I)) antagonist.

In another preferred embodiment, the antagonist of CCR2 function is an agent which can inhibit the binding of a (i.e., one or more) CCR2 ligand to CCR2, such as an agent which can inhibit the binding of human MCP-1, MCP-2, MCP-3, MCP-4 and/or MCP-5 to human CCR2.

In particularly preferred embodiment, the antagonist of CCR2 function is an agent which can bind to CCR2 and thereby inhibit the binding of a (i.e., one or more) CCR2 ligand to CCR2 (e.g., human CCR2).

Methods of Therapy

As used herein, the term “graft” refers to organs and/or tissues which can be obtained from a first mammal (or donor) and transplanted into a second mammal (a recipient), preferably a human. The term “graft” encompasses, for example, skin, eye or portions of the eye (e.g., cornea, retina, lens), muscle, bone marrow or cellular components of the bone marrow (e.g., stem cells, progenitor cells), heart, lung, heart-lung (e.g., heart and a single lung, heart and both lungs), liver, kidney, pancreas (e.g., islet, β cells), parathyroid, bowel (e.g., colon, small intestine, duodenum), neuronal tissue, bone and vasculature (e.g., artery, vein). A graft can be obtain from a suitable mammal (e.g., human, pig, baboon, chimpanzee), or under certain circumstances a graft can be produced in vitro by culturing cells, for example, embryonal cells, fetal cells, skin cells, blood cells and bone marrow cells which were obtained from a suitable mammal. A graft is preferably obtained from a human.

The graft can be obtained from a genetically modified animal or can be modified (e.g., genetically, chemically, physically) using any suitable method. In one embodiment, a modified graft having reduced capacity to express a ligand for CCR2 (e.g., MCP-1, MCP-2, MCP-3, MCP-4, MCP-5), relative to a suitable control (e.g., an unmodified or wild type graft), is transplanted. Such a graft can, for example, carry a targeted mutation in a gene encoding a CCR2 ligand. Targeted mutations can be produced using a variety of suitable methods. For example, a targeted mutation can be introduced into the genome of embryonic stem cells or zygotes using standard techniques. The resulting mutant cells can develop into animals carrying the targeted mutation (e.g., heterozygous or homozygous). For example, pigs or other animals which express human MHC antigens and which are homozygous for a targeted mutation in a gene encoding a CCR2 ligand (e.g., MCP-1, MCP-2, MCP-3, MCP-4, MCP-5) can be created. The organs from such animals (xenografts) can be transplanted into a human.

An “allograft”, as the term is used herein, refers to a graft comprising antigens which are allelic variants of the corresponding antigens found in the recipient. For example, a human graft comprising an MHC class II antigen encoded by the HLA-DRB1*0401 allele is an allograft if transplanted into a human recipient whose genome does not comprise the HLA-DRB1*0401 allele.

In one embodiment, the method of inhibiting (reducing or preventing) graft rejection comprises administering an effective amount of an (i.e., one or more) antagonist of CCR2 function to a recipient of a graft. In another embodiment, the method of inhibiting graft rejection comprises administering an effective amount of an antagonist of CCR2 function to a recipient of an allograft. In a preferred embodiment, the method comprises administering an effective amount of an antagonist of CCR2 function to a recipient of a cardiac allograft.

In another embodiment, the antagonist of CCR2 flnction is selected from the group consisting of small organic molecules, natural products, peptides, peptidomimetics and proteins, wherein said proteins are not chemokines or mutants or analogues thereof.

In a preferred embodiment, the invention provides a method for inhibiting (reducing or preventing) graft rejection comprising administering to a graft recipient an effective amount of an antagonist of CCR2 function and an effective amount of an (i.e., one or more) additional therapeutic agent, preferably, an immunosuppressive agent. Advantageously, the rejection-inhibiting effects of CCR2 antagonists and immunosuppressive agents can be additive or synergistic, and can result in permanent engraftment.

A further benefit of co-administration of a CCR2 antagonist and an immunosuppressive agent is that the dose of immunosuppressive agent required to inhibit graft rejection can be reduced to sub-therapeutic levels (e.g., a dose that does not inhibit graft rejection when administered as the sole therapeutic agent). The ability to reduce the dose of the immunosuppressive agent can greatly benefit the graft recipient as many immunosuppressive agents have severe and well-known side effects including, for example, increased incidence of infection, increased incidence of certain malignancies, diabetes mellitus, neurotoxicity, nephrotoxicity, hyperlipidemia, hypertension, hirsutism, gingival hyperplasia, impaired wound healing, lymphopenia, jaundice, anemia, alopecia and thrombocytopenia (Spencer, C. M., et al., Drugs, 54(6):925-975 (1997); Physicians Desk Reference, 53^{rd} Edition, Medical Economics Co., pp. 2081-2082 (1999)).

The term “immunosuppressive agent”, as used herein, refers to compounds which can inhibit an immune response. The immunosuppressive agent used in the invention can be a novel compound or can be selected from the compounds which are known in the art, for example, calcineurin inhibitors (e.g., cyclosporin A, FK-506), IL-2 signal transduction inhibitors (e.g., rapamycin), glucocorticoids (e.g., prednisone, dexamethasone, methylprednisolone, prednisolone), nucleic acid synthesis inhibitors (e.g., azathioprine, mercaptopurine, mycophenolic acid) and antibodies to lymphocytes or antigen-binding fragments thereof (e.g., OKT3, anti-IL2 receptor). Novel immunosuppressive agents can be identified by those of skill in the art by suitable methods, for example, screening compounds for the capacity to inhibit antigen-dependent T cell activation.

The immunosuppressive agent used for co-therapy (e.g., co-administration with an antagonist of CCR2 function) is preferably a calcineurin inhibitor. More preferably the immunosuppressive agent used for co-therapy is cyclosporin A.

When the graft is bone marrow, cells (e.g., leukocytes) derived from the graft can mount an immune response directed at the recipient's organs and tissues. Such a condition is referred to in the art as graft versus host disease (GVHD). Administration of an antagonist of CCR2 function with or without an additional therapeutic agent (e.g., immunosuppressive agent, hematopoietic growth factor) can inhibit GVHD. Accordingly, in another embodiment, the invention provides a method of inhibiting (reducing or preventing) GVHD in a bone marrow graft recipient comprising administering an effective amount of an antagonist of CCR2 function. In an additional embodiment, the method of inhibiting GVHD comprises the administration of an effective amount of an antagonist of CCR2 function and an effective amount of one or more additional therapeutic agents, for example, an immunosuppressive agent.

In another embodiment, the method of inhibiting GVHD comprises the administration of an effective amount of an antagonist of CCR2 function, which is selected from the group consisting of small organic molecules, natural products, peptides, peptidomimetics and proteins, wherein said proteins are not chemokines or mutants or analogues thereof.

The invention further relates to the use of an antagonist of CCR2 function for the manufacture of a medicament for inhibiting graft rejection (e.g., acute rejection, chronic rejection) as described herein. The invention also relates to a medicament for inhibiting graft rejection (e.g., acute rejection, chronic rejection) wherein said medicament comprises an antagonist of CCR2 function.

A “subject” is preferably a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

An effective amount of the antagonist of CCR2 function can be administered to a subject to inhibit (reduce or prevent) graft rejection. For example, an effective amount of the antagonist of CCR2 function can be administered before, during and/or after transplant surgery or other medical procedure for introduction of a graft to a recipient (e.g., transfusion).

When co-administration of an antagonist of CCR2 function and an additional therapeutic agent is indicated or desired for inhibiting graft rejection, the antagonist of CCR2 function can be administered before, concurrently with or after administration of the additional therapeutic agent. When the antagonist of CCR2 function and additional therapeutic agent are administered at different times, they are preferably administered within a suitable time period to provide substantial overlap of the pharmacological activity (e.g., inhibition of CCR2 function, immunosuppression) of the agents. The skilled artisan will be able to determine the appropriate timing for co-administration of an antagonist of CCR2 function and an additional therapeutic agent depending on the particular agents selected and other factors.

An “effective amount” of a CCR2 antagonist is an amount sufficient to achieve a desired therapeutic and/or prophylactic effect, such as an amount sufficient to inhibit graft rejection. For example, an effective amount is an amount sufficient to inhibit a (i.e., one or more) function of CCR2 (e.g., CCR2 ligand-induced leukocyte migration, CCR2 ligand-induced integrin activation, CCR2 ligand-induced transient increase in the concentration of intracellular free calcium [Ca²⁺]ᵢ and/or CCR2 ligand-induced secretion (e.g. degranulation) of proinflammatory mediators), and thereby, inhibit graft rejection. An “effective amount” of an additional therapeutic agent (e.g., immunosuppressive agent) is an amount sufficient to achieve a desired therapeutic and/or prophylactic effect (e.g., immunosuppression).

The amount of agent (e.g., CCR2 antagonist, additional therapeutic agent) administered to the individual will depend on the characteristics of the individual, such as general health, age, sex, body weight and tolerance to drugs as well as the degree, severity and type of rejection. The skilled artisan will be able to determine appropriate dosages depending on these and other factors. Typically, an effective amount can range from about 0.1 mg per day to about 100 mg per day for an adult. Preferably, the dosage ranges from about 1 mg per day to about 100 mg per day. Antibodies and antigen-binding fragments thereof, particularly human, humanized and chimeric antibodies and antigen-binding fragments can often be administered less frequently than other types of therapeutics. For example, an effective amount of such an antibody can range from about 0.01 mg/kg to about 5 or 10 mg/kg administered daily, weekly, biweekly, monthly or less frequently.

The agent (e.g., CCR2 antagonist, additional therapeutic agent) can be administered by any suitable route, including, for example, orally (e.g., in capsules, suspensions or tablets) or by parenteral administration. Parenteral administration can include, for example, intramuscular, intravenous, intraarticular, intraarterial, intrathecal, subcutaneous, or intraperitoneal administration. The agent (e.g., CCR2 antagonist, additional therapeutic agent) can also be administered orally (e.g., dietary), transdermally, topically, by inhalation (e.g., intrabronchial, intranasal, oral inhalation or intranasal drops) or rectally. Administration can be local or systemic as indicated. The preferred mode of administration can vary depending upon the particular agent (e.g., CCR2 antagonist, additional therapeutic agent) chosen, however, oral or parenteral administration is generally preferred.

The agent (e.g., CCR2 antagonist, additional therapeutic agent) can be administered as a neutral compound or as a salt. Salts of compounds containing an amine or other basic group can be obtained, for example, by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium, potassium and the like.

The antagonist of CCR2 function can be administered to the individual as part of a pharmaceutical composition for inhibition of graft rejection (e.g., acute rejection, chronic rejection) comprising a CCR2 antagonist and a pharmaceutically or physiologically acceptable carrier. Pharmaceutical compositions for co-therapy can comprise an antagonist of CCR2 function and one or more additional therapeutic agents. An antagonist of CCR2 function and an additional therapeutic agent can be components of separate pharmaceutical compositions which can be mixed together prior to administration or administered separately. Formulation will vary according to the route of administration selected (e.g., solution, emulsion, capsule). Suitable pharmaceutical or physiological carriers can contain inert ingredients which do not interact with the antagonist of CCR2 function and/or additional therapeutic agent. Standard pharmaceutical formulation techniques can be employed, such as those described in Remington's Pharmaceutical Sciences, Mack Publishing Company, Easton, PA. Suitable carriers for parenteral administration include, for example, sterile water, physiological saline, bacteriostatic saline (saline containing about 0.9% benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

The present invention will now be illustrated by the following Examples, which are not intended to be limiting in any way.

### EXAMPLES

### Example 1

### CCR2 Targeting in Cardiac Transplantation

Methods

Mice

CCR2 KO mice (also referred to as CCR2−/−)(B6/129 F6, H-2^{b}), which are homozygous for a targeted gene disruption of the gene encoding CCR2 (Kuziel, W. A. et al., Proc. Natl. Acad. Sci. U.S.A., 94:12053-12058 (1997)) were provided by William Kuziel (Austin, Tex.). All other mice were obtained from Jackson Laboratory (Bar Harbor, Me.). These included donor strains (BALB/c, H-2^{d}) and control recipients (C57BL/6, H-2^{b}; B6/129). BALB/c differs from both C57BL/6 and B6/129 at both class I and class II major histocompatibility complex (MHC) loci.

Mouse cardiac allografting (Mottram, P. L. et al., Transplantation 59:559-565 (1995); Hancock, W. W., et al., Proc. Natl. Acad. Sci (USA), 93:13967-13972 (1996)) was performed with the aid of an operating microscope (Nikon, 4× to 38× magnification) under clean conditions.

Preparation of the Donor Heart

Donor mice were anesthetized with Nembutal (50 mg/10 g body weight) and Atropine sulfate (0.17 mg/100 g body weight) i.p.; additional anaesthesia with Methoxyflurane supplementation was administered via a face mask as required during the procedure. Mice were shaved and cleansed with 70% alcohol. A midline abdominal incision was made in the donor animal and 1 ml of a 10% solution of heparin in saline was injected into the inferior vena cava. The incision was then extended cephalic to open the chest through a median stemotomy. The thorax was opened. The inferior vena cava was ligated with 6-0 silk and divided inferior to the tie. The superior vena cava was then similarly ligated and divided superior to the tie. The aorta and pulmonary artery were separated and divided as far distally as possible. At this point, blood was evacuated from the heart by applying pressure with applicator sticks. The aorta was transected just proximal to the brachiocephalic artery and the main pulmonary artery transected just proximal to its bifurcation. The pulmonary veins were then ligated and divided en mass and the heart placed in iced saline.

Preparation of the Recipient

After being anesthetized in the same way as the donor, the recipient was brought under the microscope, a midline abdominal incision was made, and segments of the aorta and vena cava below the renal vessels were dissected free, but not separated from each other, over a length of about 2 mm. A clamp was placed on the proximal aorta and vena cava, and a distal tie of 6-0 silk was placed around both the aorta and vena cava in preparation for later occlusion of the vessels.

Transplantation of the Heart

The tie that had been placed around the distal aorta and vena cava was secured by means of a single knot. An aortotomy and a venotomy in the vena cava were made adjacent to one another. The donor heart was then removed from the chilled saline, and the donor aorta and pulmonary artery were joined end-to-side to the recipient aorta and vena cava, respectively, with running suture, using 10-0 tipped with a BV-3 needle. Since the anastomoses were done adjacent to one another, the side of the pulmonary artery-cava suture line next to the aortic anastomosis was sutured from the inside with an everting running suture. During this period, chilled saline was dripped on the ischemic heart at frequent intervals. After the completion of the anastomoses, the inferior vascular occluding tie was released first, thus filling the inferior vena cava and donor pulmonary artery with recipient venous blood. Upon release of the proximal occluding tie, the aorta and coronary arteries of the transplant were perfused with oxygenated recipient blood. Blood loss was minimized by gradual release of the proximal tie. Warm saline was used externally to warm the heart immediately after establishing coronary perfusion. With warming and coronary perfusion, the heart began to fibrillate and usually within a few minutes it reverted spontaneously to a sinus rhythm. Occasionally, cardiac massage was required to re-establish a normal beat. The intestines were placed carefully back into the abdominal cavity around the auxiliary heart, and the abdomen was closed with a single running suture to all layers (saline with antibiotic was used to wash the peritoneal cavity as needed). The mouse was then placed in a constant temperature at 35° C. for recovery from anesthesia.

Monitoring of Allograft Survival

Cardiac allograft survival was monitored twice daily by palpation of ventricular contractions through the abdominal wall (Mottram, P. L. et al., Transplantation, 59:559-565 (1995)), rejection was defined as the day of cessation of palpable heartbeat, and was verified by autopsy (Gerard, C, et al., J. Clin Invest., 100:2022-2027 (1997); Mottram, P. L.,et al., Transplantation, 59:559-565 (1995)). Once cardiac graft function ceased, mice were anesthetized as above, and grafts were surgically excised, subdivided into portions for (a) formalin fixation, paraffin embedding and subsequent light microscopy examination, or (b) snap-frozen in liquid nitrogen and stored at −70° C. until processed for immunohistology or RNAse protection assays.

Immunopathology

For histology, paraffin sections were stained with hematoxylin and eosin (H&E) to evaluate graft morphology, and with Weigert's elastin stain so as to examine the extent of intimal proliferation in penetrating branches of myocardial arteries (a key feature of transplant arterioscelerosis). (Gerard, C, et al., J. Clin Invest. 100:2022-2027 (1997); Mottram, P. L., et al., Transplantation 59:559-565 (1995)). Chemokine and chemokine receptor mRNA expression was determined using RNAse protection assay kits (Pharmingen, San Diego, Calif.).

Results

Allograft survival data (mean+SD) are summarized in Table 1 (using 6-10 animals/group)

TABLE 1
Effect of CCR2 KO on mouse cardiac allograft survival

| | | | Survival | |
|---|---|---|---|---|
| | Strains | MHC | (mean ± SD, | |
| # | (Donor → Recipient) | mismatch | days) | probability |
| 1 | BALB/c → C57BL/6 | Class I & II | 7.3 ± 0.5 | |
| 2 | BALB/c → CCR2 KO | Class I & II | 14.3 ± 0.8 | p < 0.01 vs #1 |

p values were determined by the Mann-Whitney U test.

The results presented in Table 1 demonstrate that CCR2⁺cells contribute to the pathogenesis of allograft rejection. Disruption of CCR2 function in a complete MHC mismatch significantly prolonged allograft survival (group 1 vs. 2).

CCR2 KO mice do not have a general defect in cellular immunity and mount normal T cell responses in response to mitogen or antigen (e.g., mixed lymphocyte response).

### Example 2

### CCR2 Targeting Inhibits Chronic Rejection in Cardiac Allograft Recipients

Administration of anti-CD 154 monoclonal antibody (mAb) can prolong the survival of allografts in murine models. However, the extended survival of grafts in anti-CD154 treated animals is complicated by the development of chronic rejection with arteriosclerosis of the graft-associated vasculature (see, for example, Ensminger, S. M. et al., Transplantation 69:2609-2612 (2000), Billings J. S. et al., Transplant Proc. 33:323 (2001)). The effect of disrupting CCR2 function on the development of chronic rejection was assessed by monitoring cardiac allografts in CCR2−/− or CCR2+/+recipients that received anti-CD 154 monoclonal antibody therapy.

Methods

Cardiac allografts derived from Balb/c donors were transplanted into CCR2−/− recipients (on C57BL/6 background) or CCR2+/+recipient control mice (also on C57BL/6 background) as described in Example 1.

Immunosuppression

Anti-CD154 mAb (BioExpress, West Lebanon, N.H.) was administered to CCR2−/− or CCR2+/+allograft recipients (12/group); 200 μg by intraperitoneal or intravenous injection on day 0 (time of transplantation).

Monitoring of chronic rejection. Cardiac allograft survival was monitored twice daily by palpation of ventricular contractions through the abdominal wall. Thirty days after transplantation, six animal from each group were sacrificed, the allografts were removed, fixed in formalin, embedded in paraffin and sections counterstained with Weigert's elastin stain. The remaining animals were sacrificed sixty days after transplantion, grafts were removed, fixed, embedded in paraffin and counterstained with Weigert's elastin stain. The sections were then examined morphologically to determine the extent of transplant-associated arteriosclerosis. Transplant-associated arteriosclerosis was quantified using a scoring system based upon the extent of intimal expansion and resultant occlusion of graft blood vessels (<5% occlusion (0); 5-20% occlusion (1); 21-40% occlusion (2); 41-60% occlusion (3); 61-80% occlusion (4); or 81-100% occlusion (5) (Murphy et al., Transplantation 64:14-19 (1997))). Results The administration of anti-CD 154 monoclonal antibody prolonged the survival of cardiac allografts in CCR2+/+recipients and in CCR2−/− recipients, thereby allowing the development of graft-associated arteriosclerosis, a hallmark of chronic rejection, to be assessed.

Results of scoring of vessels within cardiac allografts removed from recipients thirty days after transplantation (6 grafts/group) and statistical evaluation (Mann-Whitney U test) are presented in Table 2.

TABLE 2
Effect of CCR2 KO (CCR2−/−) on development of transplant arteriosclerosis

| | Strains | | Vessels | Vessel Score | |
|---|---|---|---|---|---|
| # | (Donor → Recipient) | Therapy | Examined | (mean ± SD) | p value |
| 1 | Balb/c → | anti-CD154 | 45 | 2.3 ± 0.3 | |
| | C57BL/6 (CCR2 +/+) | | | | |
| 2 | Balb/c → | anti-CD154 | 45 | 0.2 ± 0.1 | p < 0.001 |
| | C57BL/6 (CCR2−/−) | | | | vs. #1 |

The key points arising from Table 2 are:

CCR2⁺cells contribute to the pathogenesis of chronic allograft rejection, and disruption of CCR2 function inhibits the development of transplant arteriosclerosis.

Similar results were obtained when grafts removed at day sixty were analyzed. Specifically, transplant arteriosclerosis and chronic rejection were dramatically inhibited in CCR2−/− recipient mice in comparison to CCR2+/+recipient mice.

While this invention has been particularly shown and described with references to preferred embodiments thereof, it will be understood by those skilled in the art that various changes in form and details can be made therein without departing from the spirit and scope of the invention as defined by the appended claims.

What is claimed is:

1. A method of inhibiting graft rejection comprising administering to a subject in need thereof an effective amount of an antagonist of CCR2 function.

2. The method of claim 1, wherein said graft is an allograft.

3. The method of claim 2, wherein said allograft is selected from the group consisting of kidney, liver, lung, heart-lung, pancreas, bowel and heart.

4. The method of claim 3, wherein said allograft is a heart.

5. The method of claim 1, wherein said antagonist of CCR2 function is selected from the group consisting of small organic molecules, natural products, peptides, proteins and peptidomimetics.

6. The method of claim 5, wherein said antagonist of CCR2 function is a small organic molecule.

7. The method of claim 5, wherein said antagonist of CCR2 function is a natural product.

8. The method of claim 5, wherein said antagonist of CCR2 function is a peptide.

9. The method of claim 5, wherein said antagonist of CCR2 function is a peptidomimetic.

10. The method of claim 5, wherein said antagonist of CCR2 function is a protein.

11. The method of claim 10, wherein said protein is an anti-CCR2 antibody or antigen-binding fragment thereof.

12. The method of claim 1, wherein the graft has reduced capacity to express a ligand for CCR2.

13. A method of inhibiting graft rejection comprising administering to a subject in need thereof an effective amount of an antagonist of CCR2 function and an effective amount of an immunosuppressive agent.

14. The method of claim 13, wherein said immunosuppressive agent is one or more agents selected from the group consisting of calcineurin inhibitors, glucocorticoids, nucleic acid synthesis inhibitors, and antibodies which bind to lymphocytes or antigen-binding fragments thereof.

15. The method of claim 14, wherein said immunosuppressive agent is a calcineurin inhibitor.

16. The method of claim 15, wherein said calcineurin inhibitor is cyclosporin A.

17. The method of claim 15, wherein said calcineurin inhibitor is FK-506.

18. The method of claim 14, wherein said immunosuppressive agent is a glucocorticoid.

19. The method of claim 18, wherein said glucocorticoid is prednisone or methylprednisolone.

20. A method of inhibiting graft versus host disease comprising administering an effective amount of an antagonist of CCR2 function to a recipient of a transplanted graft.

21. The method of claim 20, wherein said graft is bone marrow.

22. The method of claim 21, further comprising administering an effective amount of an immunosuppressive agent.

23. The method of claim 22, wherein said immunosuppressive agent is a calcineurin inhibitor.

24. The method of claim 23, wherein said calcineurin inhibitor is cyclosporin A or FK-506.

25. The method of claim 11, wherein said anti-CCR2 antibody or antigen-binding fragment comprises light chain complementarity determining regions (CDR1, CDR2 and CDR3) of nonhuman origin, heavy chain complementarity determining regions (CDR1, CDR2 and CDR3) of nonhuman origin, and at least a portion of an immunoglobulin of human origin, wherein said light chain complementarity determining regions and said heavy chain complementarity determining regions have the amino acid sequences set forth below:
light chain: CDR1 KSSQSLLDSDGKTFLN (SEQ ID NO:14)
CDR2 LVSKLDS (SEQ ID NO:15)
CDR3 WQGTHFPYT (SEQ ID NO:16)
heavy chain: CDR1 AYAMN (SEQ ID NO:17)
CDR2 RIRTKNNNYATYYADSVKD (SEQ ID NO:18)
CDR3 FYGNGV (SEQ ID NO:19).

26. The method of claim 25, wherein said anti-CCR2 antibody or antigen-binding fragment comprises:
a light chain variable region having an amino acid sequence selected from the group consisting of SEQ ID NO:3, SEQ ID NO:4, SEQ ID NO:5, SEQ ID NO:6 and SEQ ID NO:7; and
a heavy chain variable region having an amino acid sequence selected from the group consisting of SEQ ID NO:10, SEQ ID NO:11, SEQ ID NO:12 and SEQ ID NO:13.

27. The method of claim 26, wherein said light chain variable region has the amino acid sequence of SEQ ID NO:3, and said heavy chain variable region has the amino acid sequence of SEQ ID NO:10.

28. A method of inhibiting chronic rejection of a transplanted graft comprising administering to a subject in need thereof an effective amount of an antagonist of CCR2 function.

29. The method of claim 28, wherein said graft is an allograft.

30. The method of claim 29, wherein said allograft is selected from the group consisting of kidney, liver, lung, heart-lung, pancreas, bowel and heart.

31. The method of claim 30, wherein said allograft is a heart.

32. The method of claim 28, wherein said antagonist is an antibody or antigen- binding fragment thereof which binds CCR2.

33. The method of claim 32, wherein said antibody or antigen-binding fragment thereof binds CCR2 and inhibits the binding of a ligand to CCR2.

34. The method of claim 33, wherein said antibody or antigen-binding fragment comprises light chain complementarity determining regions (CDR1, CDR2 and CDR3) of nonhuman origin, heavy chain complementarity determining regions (CDR1, CDR2 and CDR3) of nonhuman origin, and at least a portion of an immunoglobulin of human origin, wherein said light chain complementarity determining regions and said heavy chain complementarity determining regions have the amino acid sequences set forth below:
light chain: CDR1 KSSQSLLDSDGKTFLN (SEQ ID NO:14)
CDR2 LVSKLDS (SEQ ID NO:15)
CDR3 WQGTHFPYT (SEQ ID NO:16)
heavy chain: CDR1 AYAMN (SEQ ID NO:17)
CDR2 RIRTKNNNYATYYADSVKD (SEQ ID NO:18)
CDR3 FYGNGV (SEQ ID NO:19).

35. The method of claim 34, wherein said antibody or antigen-binding fragment comprises:
a light chain variable region having an amino acid sequence selected from the group consisting of SEQ ID NO:3, SEQ ID NO:4, SEQ ID NO:5, SEQ ID NO:6 and SEQ ID NO:7; and
a heavy chain variable region having an amino acid sequence selected from the group consisting of SEQ ID NO:10, SEQ ID NO:11, SEQ ID NO:12 and SEQ ID NO:13.

36. The method of claim 35, wherein said light chain variable region has the amino acid sequence of SEQ ID NO:3, and said heavy chain variable region has the amino acid sequence of SEQ ID NO:10.

37. The method of claim 28, further comprising administering to said subject an effective amount of an immunosuppressive agent.

38. The method of claim 37, wherein said immunosuppressive agent is one or more agents selected from the group consisting of calcineurin inhibitors, glucocorticoids, nucleic acid synthesis inhibitors, and antibodies which bind to lymphocytes or antigen-binding fragments thereof.

39. The method of claim 38, wherein said immunosuppressive agent is a calcineurin inhibitor.

40. The method of claim 39, wherein said calcineurin inhibitor is cyclosporin A.

41. The method of claim 39, wherein said calcineurin inhibitor is FK-506.

42. The method of claim 38, wherein said immunosuppressive agent is a glucocorticoid.

43. The method of claim 42, wherein said glucocorticoid is prednisone or methylprednisolone.
